(19) United States
(12) Patent Application Publication
(10) Pub. No.: US 2015/0018742 A1
(43) Pub. Date: Jan. 15, 2015

(54) SELF-SECURING MEDICAL DEVICE AND METHOD

(71) Applicant: John Adam Baumgartner, Winter Park, FL (US)

(72) Inventor: John Adam Baumgartner, Winter Park, FL (US)

(21) Appl. No.: 13/938,759

(22) Filed: Jul. 10, 2013

(51) Int. Cl. A61F 13/00

(52) U.S. Cl. CPC A61F 13/00021; A61F 13/00063; A61F 2013/00119; A61F 2013/00285
USPC 602/48; 602/53

(57) ABSTRACT

In one embodiment, a medical device for containing or applying a pressure or a combination thereof to an area of skin or wound of a user is provided. The medical device includes a bandage body, a pad component disposed thereon, body, an adhesive component, and an opening adjacent to the first end of the bandage body, wherein the opening is configured to receive at least a portion of the bandage body such that when the second end of the bandage body is received within the opening, the adhesive portion of bandage body can be attached to a portion of the bandage body such that the adhesive does not contact the user.

### BACKGROUND

A wide variety of materials have been used to treat or dress different types or kinds of wounds. Typical materials that are used in the various dressings include wound contact materials and absorbent materials as well as compressive materials which are used to hold the wound contact and absorbent materials in place and which provide compressive force to the wound. Elastic fabric wraps are often used as an outer layer of compression wound dressings in order to hold the inner layers in place and to apply compression to the wound. The use of fabric elastomeric wraps; however, is not always desirable because dressings wrapped with elastic fabrics are likely to slip or shift after being applied which may result in insufficient compression being applied to the wound by the dressing or which may cause discomfort to the patient. Attempts to overcome the problems of slipping or shifting include, for example, applying a hydrocolloid adhesive to one side of the fabric wrap. The hydrocolloid adhesive is intended to stick to skin in order to prevent slippage.

Adhesives used to stick to skin in order to keep a material, a wrap, or a bandage in place can cause irritation to the skin. Direct adhesion onto the skin; therefore, is a significant problem as in some instances patients may develop a skin reaction to the components of the adhesive. Irritant contact dermatitis is a nonallergic, localized, inflammatory skin reaction to external chemical or physical agents. It does not involve the central immune system (no memory T-cell activation), and is the most commonly reported adverse reaction to many transdermal medications. There are many classifications of irritant contact dermatitis; however, with transdermal medications, signs and symptoms usually arise from: (a) damage to the corneocytes and sloughing of the cells during patch removal; (b) the blocking of intraepidermal eccrine sweat ducts or follicular ostium (or opening of the pilosebaceous units); (c) removal of oil (or lipids) from all layers of the stratum corneum, leading to increased water loss; (d) irritation caused by the drug or other components of the patch (e.g., penetration enhancers, solvents, adhesive); (e) an occlusive effect preventing water evaporation and trapping sweat, producing irritation and swelling of the stratum corneum; (f) irritant effects of residual substances such as soap, lotions, or emollients; and (g) friction produced by differential shearing forces between immobile skin beneath the patch and the surrounding mobile skin. All of these factors may cause damage to the stratum corneum, which can be cumulative with continuous or repeated use. Compromising the stratum corneum would in turn allow further penetration of irritants into the skin layers, leading to inflammation, further damage, and alterations in skin pharmacokinetics. (Skin Tolerability Associated with Transdermal Drug Delivery Systems: an Overview, Iris Ale et al. Adv. Ther (2009); 23(10):920-935).

### SUMMARY

The inventor has discovered an embodiment of a medical device including a bandage with a beneficial arrangement used to treat a wound of a patient without requiring an adhesive to come into contact with the skin of the patient. In one embodiment, a medical device for containing or applying a pressure or a combination thereof to an area of skin or wound of a user is provided. The medical device includes a bandage body including a first side and a second side, a first end and a second end, a pad component disposed on the first side of the bandage body, an adhesive component disposed on the second side of the bandage body, and an opening adjacent to the first end of the bandage body, wherein the opening is configured to receive at least a portion of the second end of the bandage body such that when the second end of the bandage body is received within the opening, the second side of the second end of the bandage body can be attached to a portion of the second side of the bandage body, wherein the attachment component is disposed between the second side of the second end of the bandage body and the portion of the second side of the bandage body attached thereto, such that the medical device attaches onto to itself.

In another embodiment, a medical device for containing or applying a pressure to, or a combination thereof, an area of skin or wound of a user is provided herein. The medical device includes a bandage body including a first side and a second side, a first end and a second end. The medical device includes a pad component disposed on the first side of the bandage body adjacent to the first end, and an adhesive component disposed on the first side of the bandage body adjacent to the second end, wherein the bandage body can be wrapped around the area of skin or wound of the user and secured to itself such that the adhesive component secures the second end of the bandage body onto a portion of the second side of the bandage, such that the bandage body adheres to itself.

In a further embodiment, a method for containing an area of skin of a user is provided herein. The method includes applying a first side of a bandage body comprising a first end and a second end and an opening adjacent to the first end, onto the area of skin of the user to be contained, such that a pad component of the bandage body contacts the area of skin of the user to be contained. The method further includes threading the second end through the opening adjacent to the first end, and folding the second end of the bandage body back onto itself, and adhering the second end of the bandage body onto a portion of the bandage body such that the bandage body contains the area of the skin of the user and no adhesive contacts the skin of the user.

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIG. 1A provides a plan view of a first side of an embodiment of the medical device.

FIG. 1B provides a view of a second side of the embodiment shown in FIG. 1A.

FIG. 2A provides a perspective view of the embodiment shown in shown in FIGS. 1A-1B wherein a portion of the medical device is inserted through another portion of the medical device.

FIG. 2B provides a perspective view of the next step in the application of the medical device shown in FIG. 2A, in one embodiment of the invention, wherein the portion inserted through the medical device is folded back on itself to secure the device onto itself.

FIG. 2C provides a perspective close up view of the connection between the ends of the medical device as shown in FIG. 2B.

FIG. 3 shows another embodiment of a medical device according to the invention, with a pad component adjacent to a first end and an adhesive adjacent to a second end.

FIG. 4A provides a plan view of a first side of a further embodiment of the medical device of the invention, wherein a pad component is provided on a first side, near a first end of the bandage body.

FIG. 4B provides a plan view of a second side of the embodiment of the medical device shown in FIG. 4A, wherein the adhesive portion is located generally central to the bandage body.

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

### Definitions

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. The modifier “about” used in connection with a quantity is inclusive of the stated value and has the meaning dictated by the context. It is to be noted that all ranges disclosed within this specification are inclusive and are independently combinable.

The terminology used herein is for the purpose of describing particular embodiments only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order or importance, but rather the terms first, second, etc., are used to distinguish one element from another.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. As a non-limiting example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 7.

“Container” as used herein refers to a housing for a compound or composition, and includes but is not limited to: capsules, tablets, ampoules, aerosol cans, sponges, syringes, vials, tubes, bottles, pouches, and strips.

As used herein, the terms “subject”, “user” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human.

The term “medicament” as used in describing the various embodiments of this invention includes a medication, drug, antibiotic, pharmaceutical, prescriptive, agent, antidote, anti-venom, hormone, stimulant, vasodilator, anesthetic, nutritional supplement, vitamin and/or mineral compound, saline solution, biological, organic compound, genetically and/or chemically modified protein and/or nucleic acids, or other composition adapted to provided to the tissue of a subject, particularly compositions useful in advancing wound healing. The medicament may include any substance or composition which may provide a therapeutic effect to a wound.

In one embodiment, a medical device for containing or applying a pressure or a combination thereof to an area of skin or wound of a user is provided. The medical device includes a bandage body including a first side and a second side, a first end and a second end, a pad component disposed on the first side of the bandage body, an adhesive component disposed on the second side of the bandage body, and an opening adjacent to the first end of the bandage body, wherein the opening is configured to receive at least a portion of the second end of the bandage body such that when the second end of the bandage body is received within the opening, the second side of the second end of the bandage body can be attached to a portion of the second side of the bandage body, wherein the attachment component is disposed between the second side of the second end of the bandage body and the portion of the second side of the bandage body attached thereto, such that the medical device attaches onto to itself. The various benefits of a medical device such as the device described herein, which attaches to itself, are numerous, and include the ability to avoid a potential allergic reaction of a user to an adhesive or attachment component. Additionally, any discomfort associated with the removal of the adhesive of the bandage or medical device from the skin or a surface of a user is eliminated with the use of the device as described herein. Furthermore, a stronger attachment component such as a stronger adhesive, for example, may be used herein to provide a more secure attachment of the medical device where the attachment component has no contact with the skin of the user.

In a further embodiment, the bandage body comprises an elastic material. In still a further embodiment, the pad component is selected from the group consisting of cotton, gauze, cotton swab, and a suitable material to seal a wound in such a way as to be able to cover and bandage a part of the user. The pad component may be formed of any material known in the art to cover over or contact a wound of a user. The pad component may include an absorbent layer. The pad component is preferably bonded to the bandage body with a suitable adhesive, wherein the adhesive is contained between the pad component and the bandage body. The pad component may further be bonded to the bandage body by any other means known in the art, such as attached or fixed to the bandage body by stitching, ultrasonic welding, needle tacking. The pad component may include a foam, woven or nonwoven material including but not limited to rayon, polyester, polyurethane, polyolefin, cellulose, cellulose derivatives, cotton, orlon, nylon, or hydrogel polymeric materials. See, e.g., U.S. Pat. No. 4,773,903 to Weisman et al. In an alternative embodiment, the pad component includes a composite material including a nonwoven polymeric matrix and a highly hydrophilic fluid absorbing material such as a polymeric absorbent fiber or particle such as a modified starch and a high molecular weight acrylic polymer containing hydrophilic group such as acrylonitrile fibers treated with alkali metal hydroxides. These absorbent materials will preferably absorb at least 25% by weight of fluid and more preferably greater than at least 95% by weight, when measured using test methods as reported in U.S. Pat. No. 4,957,795 to Riedel.

In a further embodiment, the pad component may include a container for housing a medicament.

In still a further embodiment, the attachment component may include an adhesive, Velcro®, a hook and eye attachment, or a button and button hole attachment, or a combination thereof, or any other type of attachment means known in the art.

In another embodiment, a medical device for containing or applying a pressure to, or a combination thereof, an area of skin or wound of a user is provided herein. The medical device includes a bandage body including a first side and a second side, a first end and a second end. The medical device includes a pad component disposed on the first side of the bandage body adjacent to the first end, and an adhesive component disposed on the first side of the bandage body adjacent to the second end, wherein the bandage body can be wrapped around the area of skin or wound of the user and secured to itself such that the adhesive component secures the second end of the bandage body onto a portion of the second side of the bandage, such that the bandage body adheres to itself.

The medical device may be used to apply pressure to a wound or other area of a user to reduce bleeding, in one example. This is particularly beneficial where the medical device is used on a limb or a digit of an extremity of the user where by nature of its ability to wrap around the limb, digit or extremity, additional pressure may be created and applied to a target site on the limb, digit or extremity, for example.

In a further embodiment the bandage body includes an elastic material. The bandage body may include a porous, conformable material with sufficient elasticity to enable the bandage body to be wrapped around a portion of the body of a user, for example. The porosity of the material will allow sufficient transmission of air and moisture vapor through the material. The bandage body may be formed of any material known in the art, including, but not limited to elastic materials. The bandage body may be comprised of, in whole or in part, a material made of melt blown microfiber webs, for example. The melt blown microfiber webs may be composed of a variety of well known thermoplastic elastomers including polyurethane, p, styrene-isoprene block copolymer, styrene-butadiene block copolymer, (KRATON polymer, Shell Oil Company, Belpre, Ohio) and blends of these elastomers with polyolefins such as polypropylene and polyethylene. In addition, the melt blown microfiber webs may include, but are not limited to, staple fibers, such as rayon, polyester, nylon, cotton, LANSEAL fiber, cellulose, or polypropylene fibers, to provide a blend of elastomeric and staple fibers. Other suitable absorbent materials include composite materials such as nonwoven polymeric matrices combined with highly hydrophilic fluid absorbing materials. Highly hydrophilic fluid absorbing materials include polymeric absorbent fibers or particles, such as, but not limited to modified polysaccharides, modified polyurethanes and high molecular weight acrylic polymers containing hydrophilic groups. A preferred highly hydrophilic fluid absorbing material is acrylonitrile fibers treated with alkali metal hydroxides. A commercially available hydrogel polymeric material is distributed under the tradename LANSEAL fiber (Japan Exlan Co., Ltd., Osaka, Japan). These types of composite absorbent materials are readily prepared using well known methods such as the method reported in U.S. Pat. No. 4,957,795 to Riedel.

Typically, the absorbent material includes one or more layers of a nonwoven, melt blown absorbent fiber which provides loft to the material and which absorbs liquids. The surface of the absorbent material which contacts the wound may additionally be treated or modified so that it will not adhere to the wound. For example, the absorbent material may be covered with a variety of commercially available wound contact materials such as TEGAPORE woven nylon web, TEGADERM polyurethane film or TEGASORB hydrocolloid (all available from 3M, St. Paul, Minn.) as well as other well known related materials. Preferably, the absorbent pad includes a coagulant, enhancing clot formation in the wound and improving bleeding control properties of the medical device. Materials which may be sterilized, including radiation sterilized, may be used to form at least a portion of the medical device herein, for example.

As used herein, the term “coagulant” is defined as any agent that promotes clotting, or coagulation of blood, which may be safely applied to an open wound. A non-limiting example of such a coagulant material comprising gelatin, thrombin and calcium is described in U.S. Pat. No. 6,045,570 to Epstein, et al. This coagulant may included on or in the pad component of the device, in one embodiment.

In a further embodiment, the pad component of the device is selected from the group consisting of cotton, gauze, cotton swab, and a suitable material to seal a wound in such a way as to be able to cover and bandage a part or wound of the user. In further embodiments, the pad component may include a medicament embedded therein or as a layer on the surface, alternatively, or in addition, the pad component may include a container for housing the medicament, such as the coagulant described above.

In a further embodiment, a method for containing an area of skin of a user is provided herein. The method includes applying a first side of a bandage body comprising a first end and a second end and an opening adjacent to the first end, onto the area of skin of the user to be contained, such that a pad component of the bandage body contacts the area of skin of the user to be contained. The method further includes threading the second end through the opening adjacent to the first end, and folding the second end of the bandage body back onto itself, and adhering the second end of the bandage body onto a portion of the bandage body such that the bandage body contains the area of the skin of the user and no adhesive contacts the skin of the user. This embodiment allows a user to apply the medical device with one hand, which can be beneficial when, for example, the device is being applied to a portion of a hand or arm of the user or in instances where one upper extremity of a user is unavailable for use in application of the device.

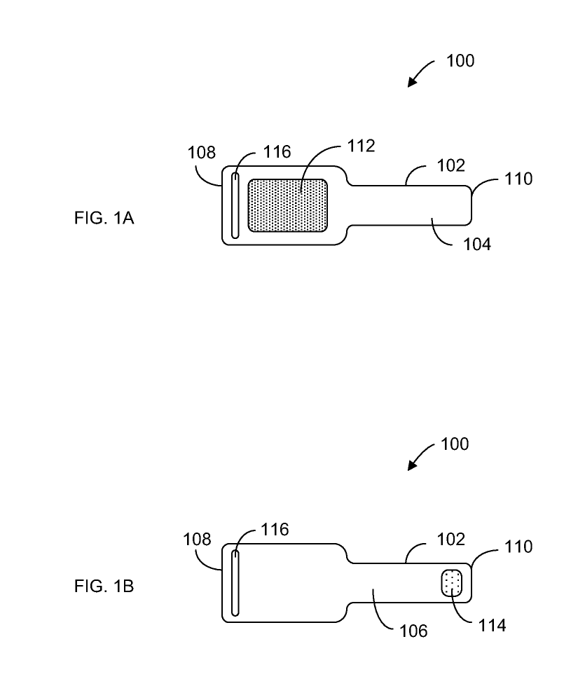

Turning to the Figures, FIG. 1A provides a plan view of a first side 104 of an embodiment of the medical device 100. A bandage body 102 is provided with a pad component 112 disposed on the first side 104 adjacent to the first end 108 of the medical device 100. An opening 116 is provided between the first end 108 and the pad component 112. A second end 110 of the device 100 is also provided. In FIG. 1B, a view of a second side 106 of the medical device 100 provided in FIG. 1A is shown. An attachment component 114 is disposed adjacent to the second side 110 of the medical device 100. The opening 116, the pad component 112, and the attachment component 114 may be provided in different positions or locations on the bandage body 102 than those shown in FIG. 1A. The orientation of the components shown in FIGS. 1A-1B is provided as a non-limiting example, only. In one embodiment, for example, the pad component 112 or the attachment component 114 can be disposed between ½ millimeter and 5 centimeters from an end of the device 100. In another embodiment, the pad component 112 or the attachment component 114 can be disposed between 1 centimeter and 3 centimeters from an end of the device 100, for example. Those of ordinary skill in the art would readily recognize that medical devices having multiple attachment components and pad components than those shown in the drawings herein are also provided by the present invention.

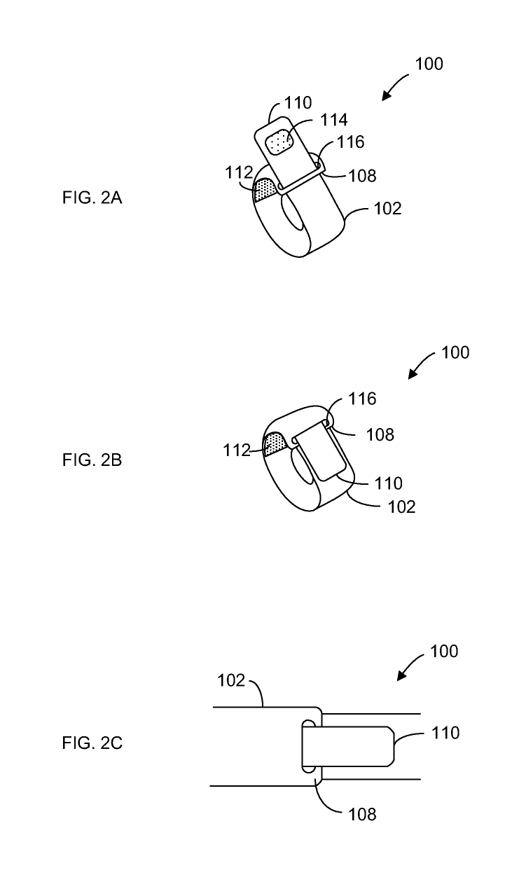

FIG. 2A provides a perspective view of the embodiment of the device 100 in FIGS. 1A-1B wherein a portion of the bandage body 102 (i.e., second end 110) is inserted through the opening 116 near the first end 108 of the device 100. The opening 116 may be disposed between 1 millimeter and 5 centimeters from the first end 108 of the device 100, in one embodiment. In another embodiment, the opening 116 may be disposed between 1 centimeter and 3 centimeters from the first end 108 of the device 100. In another embodiment, the pad component 112 may be disposed on between ⅛ and ⅞ of the surface of the bandage body 102. A ratio of the pad component 112 portion of the device 100 to the remainder (non-pad) portion of the device may be 1:1 or 1:10, or any ratio there between including 1:2, 1:3, 1:4, 1:5, 1:6, 1:7, 1:8, 1:9, for example. Alternatively, the ratio of the pad component 112 portion of the device 100 to the remainder (non-pad) of the device 100 may be 10:1 to 2:1 or any ratio there between including 9:1, 8:1, 7:1, 6:1, 5:1, 4:1, 3:1, for example.

FIG. 2B provides a perspective view of an embodiment of the bandage wherein the attachment component 114 is secured to a portion of the bandage body 102 once the second end 110 is passed through the opening 116 in the medical device 100. This allows the medical device 100 to be attached to itself instead of a user's body or skin. FIG. 2C provides a zoomed in view of the portion of the device 100 where the second end 110 attaches to a portion of the bandage body 102 as also shown in FIG. 2B. The embodiment shown in FIGS. 2A-C greatly reduces the possibility of applying the device 100 too tightly to a user's appendage, for example. In this embodiment, because the bandage body 102 is inserted through its opening 116 and folds back over itself for attachment via the attachment component 114, placing the device 100 too tightly on a user would cause the bandage body 102 to tear, for example, near the portion of the bandage body 102 which traverses the opening 116 before the attachment component 114 would be attached to the bandage body 102.

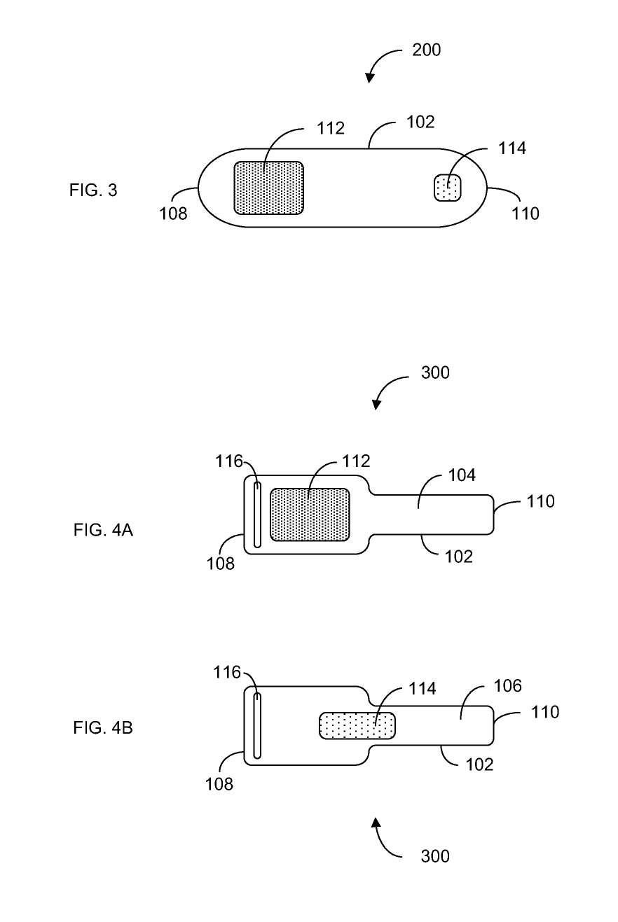

FIG. 3 shows a further embodiment 200 of a medical device wherein the pad component 112 is adjacent to a first end 108 and the attachment component 114 is adjacent to a second end 110. In this embodiment of the medical device 200, the pad component 112 and attachment component 114 are located on the same side of the bandage body 102. The bandage body 102 can be wrapped around itself and the attachment component 114 can be attached to the bandage body 102 on the side of the device 200 not shown in the Figure.

In a further embodiment, the device 100 may include one or more LED illuminated side emitting fiber optic strands or LEDs embedded in the bandage body 102 or the pad component 112 of the device 100. The LEDs may be coupled to a DC power source on the device 100 or connected thereto in a manner known in the art. The LEDs may provide a wound sanitizing and/or healing function when placed next to or on a wound of a user. Upon activation of said LEDs, the wound or surface of the user being illuminated is effectively sanitized. In one embodiment, the device 100 may include a peel-off backing, for example, which contains an embedded magnetic material which when removed activates a magnetically activated switch in the bandage 102 initiating an illumination cycle. Electronic circuitry may be traced on a circuit board, which may also be embedded in the device 100, in one embodiment, wherein a time of activation is controlled by a digital timer on the circuit board and the digital timer is a microprocessor controller with a preprogrammed timing algorithm, for example.

In a further embodiment, FIG. 4A provides a plan view of a first side 104 of the medical device 300 according to the invention, wherein the pad component 112 is provided on the first side 104, near a first end 108 of the bandage body 102. The second side 106 of the device 300 shown in FIG. 4A includes an attachment component 114 disposed generally centrally on the bandage body 102 between the first end 108 and the second end 110 as shown in FIG. 4B. In this embodiment, the bandage body 102 can be wrapped around a portion, for example, an appendage of a user, and secured onto itself such that the second end 110 is inserted through the opening 116 near the first end 108, which is then wrapped back onto itself so that the second end 110 overlaps the attachment component 114 of the device 300. As aforementioned, the attachment component 114 may be provided in varying locations on the bandage body 102, the positions of the components as provided in the Figures submitted herewith are for example only and are not intended to be limiting. The opening 116 may also be provided in different positions on the device 100, and may be in varying shapes, including, but not limited to rectangular, circular, and square, and other such shapes which would create an opening to allow passage of material (i.e., a portion of bandage body 102) there through.

It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A medical device for containing or applying a pressure, or a combination thereof, to an area of skin or wound of a user, said medical device comprising:
a bandage body comprising a first side and a second side, a first end and a second end;
a pad component disposed on the first side of the bandage body;
an attachment component disposed on the second side of the bandage body; and
an opening, wherein said opening is adjacent to the first end of the bandage body, said opening is configured to receive at least a portion of the second end of the bandage body such that when the second end of the bandage body is received within the opening, the second side of the second end of the bandage body can be attached to a portion of the second side of the bandage body, wherein the attachment component is disposed between the second side of the second end of the bandage body and the portion of the second side of the bandage body attached thereto, such that the medical device attaches onto to itself.

2. The medical device of claim 1, wherein the bandage body comprises an elastic material.

3. The medical device of claim 1, wherein the pad component is selected from the group consisting of cotton, gauze, cotton swab, and a suitable material to seal a wound in such a way as to cover and bandage a part of the user.

4. The medical device of claim 1, wherein the pad component further comprises a container for housing a medicament.

5. The medical device of claim 1, wherein the attachment component comprises an adhesive, Velcro®, a hook and eye attachment, or a button and button hole attachment, or a combination thereof.

6. A medical device for containing or applying a pressure to, or a combination thereof, an area of skin or wound of a user, said medical device comprising:
a bandage body comprising a first side and a second side, a first end and a second end;
a pad component disposed on the first side of the bandage body adjacent to the first end; and
an adhesive component disposed on the first side of the bandage body adjacent to the second end;
wherein the bandage body can be wrapped around the area of skin or wound of the user and secured to itself such that the adhesive component secures the second end of the bandage body onto a portion of the second side of the bandage, such that the bandage body adheres to itself.

7. The medical device of claim 6, wherein the bandage body comprises an elastic material.

8. The medical device of claim 6, wherein the pad component is selected from the group consisting of cotton, gauze, cotton swab, and a suitable material to seal a wound in such a way as to be able to cover and bandage a part of the user.

9. The medical device of claim 6, wherein the pad component comprises a medicament.

10. The medical device of claim 6, wherein the pad component further comprises a container for housing a medicament.

11. A method for containing an area of skin of a user, comprising:
applying a first side of a bandage body comprising a first end and a second end and an opening adjacent to the first end, onto the area of skin of the user to be contained such that a pad component of the bandage body contacts the area of skin of the user to be contained;
inserting the second end through the opening adjacent to the first end; and
folding the second end of the bandage body back onto itself, and adhering the second end of the bandage body with an attachment component, to a portion of the bandage body such that the bandage body contains the area of the skin of the user and the attachment component is not in contact with the skin of the user.
